(19) United States
(12) Patent Application Publication
(10) Pub. No.: US 2025/0228821 A1
(43) Pub. Date: Jul. 17, 2025

(54) N-DESMETHYL RUBOXISTAURIN AS A KINASE INHIBITOR

(71) Applicant: 4M Therapeutics Inc., Skillman, NJ (US)

(72) Inventors: Pablo Lapuerta, Skillman, NJ (US); Daniel E. Levy, San Mateo, CA (US)

(73) Assignee: 4M Therapeutics Inc., Skillman, NJ (US)

(21) Appl. No.: 18/853,006

(22) PCT Filed: Mar. 31, 2023
(86) PCT No.: PCT/US2023/065205

§ 371 Date: Sep. 30, 2024

Related U.S. Application Data
(60) Provisional application No. 63/362,293, filed on Mar. 31, 2022.

(51) Int. Cl. A61K 31/407; A61K 31/19; A61K 31/4439; A61K 31/454; A61K 31/46; A61K 31/495; A61K 31/496; A61K 31/519; A61K 31/53; A61K 31/55; A61K 31/5513; A61K 31/554; A61K 33/00

(52) U.S. Cl. CPC A61K 31/407; A61K 31/19; A61K 31/4439; A61K 31/454; A61K 31/46; A61K 31/495; A61K 31/496; A61K 31/519; A61K 31/53; A61K 31/55; A61K 31/5513; A61K 31/554; A61K 33/00

(57) ABSTRACT

Aspects of this invention are related to the use of N-desmethyl ruboxistaurin and pharmaceutically acceptable formulations thereof to modulate GSK3|3 signaling. Some aspects of the invention relate to the use of N-desmethyl ruboxistaurin to inhibit protein kinase C. Some aspects of the invention provide methods of using N-desmethyl ruboxistaurin in the treatment of subjects having a neurological disease and/or psychiatric disorder, including Alzheimer's disease, frontotemporal dementia, behavioral complications of dementia, bipolar disorder, depression, schizophrenia, Parkinson's disease, or neuroinflammation. Some aspects of this invention provide methods of using N-desmethyl ruboxistaurin in treating conditions associated with diabetes mellitus or its complications, or ischemia, inflammation, pulmonary hypertension, congestive heart failure, cardiovascular disease, dermatological disease, or cancer. Some aspects of the invention relate to the use of N-desmethyl ruboxistaurin as an alternative to ruboxistaurin, with a better pharmacokinetic profile, lower potential for prolongation of the electrocardiogram QT interval, and/or a lower potential for adverse drug interactions. In some embodiments, N-desmethyl ruboxistaurin is administered in combination with lithium or other treatments for bipolar disorder.

### CROSS-REFERENCE TO RELATED APPLICATION

The present application claims priority benefit under 35 U.S.C. § 119(e) to U.S. Provisional Patent Application Ser. No. 63/362,293 filed Mar. 31, 2022, the disclosure of which is incorporated herein by reference.

### FIELD OF THE INVENTION

Aspects of this invention are related to methods for treating neurological disease or psychiatric disorders, including Alzheimer's disease frontotemporal dementia, behavioral complications of dementia, bipolar disorder, depression, schizophrenia, Parkinson's disease, or neuroinflammation, and for treating diabetes mellitus and its complications, or ischemia, inflammation, pulmonary hypertension, congestive heart failure, cardiovascular disease, dermatological disease, cancer, or GM2 gangliosidosis, or other conditions where ruboxistaurin is clinically useful.

### BACKGROUND OF THE INVENTION

Ruboxistaurin has been shown to modulate GSK3 signaling and to inhibit protein kinase C.

As a GSK3 inhibitor, ruboxistaurin has been proposed as a treatment for subjects having a neurological disease and/or psychiatric disorder, including Alzheimer's disease, frontotemporal dementia, behavioral complications of dementia, bipolar disorder, depression, schizophrenia, Parkinson's disease, or neuroinflammation. Inhibitors of GSK3 are known to increase the expression of WNT proteins, thereby enhancing a pathway in regenerative medicine that has been broadly proposed to treat neurological and psychiatric disorders and reduce neuroinflammation. GSK3 inhibition or enhancement of WNT signaling has been linked to the potential treatment of type 2 diabetes and renal disorders including diabetic nephropathy, chronic kidney disease, polycystic kidney disease, and focal segmental glomerulosclerosis, and atherosclerosis, alopecia, bone and joint disorders including osteoarthritis and osteoporosis, inflammatory disorders including alcoholic hepatitis inflammatory bowel disease, and septic shock, disorders of the eye including wet age-related macular degeneration, dry age-related macular degeneration, diabetic macular edema, Fuch's dystrophy, limbal cell deficiency, dry eye, glaucoma, familial exudative vitreoretinopathy (FEVR), Norrie disease, Coats disease, retinopathy of prematurity, macular telangiectasia, retinal vein occlusion, and Sjögren's syndrome. GSK3 inhibition or enhancement of WNT signaling has been linked to the potential treatment of ear disorders including sensorineural hearing loss and conductive hearing loss, pulmonary disorders including chronic obstructive pulmonary disease (COPD) and idiopathic pulmonary fibrosis, and short bowel syndrome, and cancers including melanoma, pancreatic cancer, prostate cancer, colon cancer, and leukemia. As a GSK3 inhibitor, the use of ruboxistaurin has been proposed as a monotherapy for treating bipolar disorder, or in combination with lithium, or in combination with other bipolar disorder treatments.

As a protein kinase C inhibitor, ruboxistaurin has been proposed for treating conditions associated with diabetes mellitus, diabetic nephropathy, diabetic neuropathy, diabetic retinopathy, ischemia, inflammation, pulmonary hypertension, congestive heart failure, cardiovascular disease, dermatological disease, cancer and GM2 gangliosidosis. Protein kinase C inhibition has also been suggested to be helpful for treatment of bipolar disorder and Alzheimer's disease.

However, ruboxistaurin pharmacokinetics include a high peak to trough ratio, and ruboxistaurin has been shown to prolong the QT interval in human subjects. Further, ruboxistaurin levels can be increased by drugs that inhibit CYP3A4 metabolism.

N-desmethyl ruboxistaurin is a metabolite of ruboxistaurin.

### BRIEF SUMMARY OF THE INVENTION

Aspects of this invention are related to the use of N-desmethyl ruboxistaurin as a therapeutic agent, and a potentially safer alternative to the use of ruboxistaurin, in situations where ruboxistaurin is clinically useful.

One aspect of the invention is directed to a method of treating a disorder comprising aberrant signaling of GSK3β or protein kinase C, by administering to a subject in need thereof a therapeutically effective dose of N-desmethyl ruboxistaurin or a pharmaceutically acceptable salt thereof, or pharmaceutical composition thereof. Stated another way, the invention provides N-desmethyl ruboxistaurin or a pharmaceutically acceptable salt thereof, for use in treating a disorder characterized by aberrant signaling of GSK3β or protein kinase C, by administering to a subject in need thereof a therapeutically effective dose of the N-desmethyl ruboxistaurin or a pharmaceutically acceptable salt thereof. The method can be applied in cases where N-desmethyl ruboxistaurin is administered to a subject who 1) has never taken ruboxistaurin, or 2) has taken ruboxistaurin and has experienced adverse effects, or 3) shows a prolonged QT interval, or 4) has been shown to have high plasma levels of ruboxistaurin, or 5) has the potential to receive drugs that might interfere with the metabolism of ruboxistaurin, or 6) might require higher doses of ruboxistaurin and there is a concern for adverse effects, QT prolongation, or adverse drug interactions.

The subject can have a neurological disease and/or psychiatric disorder. The disease/disorder can be selected from Alzheimer's disease, frontotemporal dementia, behavioral complications of dementia, bipolar disorder, depression, schizophrenia, Parkinson's disease, and/or neuroinflammation.

The subject can have diabetes mellitus, diabetic neuropathy, diabetic retinopathy, diabetic nephropathy, chronic kidney disease, atherosclerosis, alopecia, osteoarthritis, osteoporosis, alcoholic hepatitis, inflammatory bowel disease, wet age-related macular degeneration, dry age-related macular degeneration, diabetic macular edema, Fuch's dystrophy, limbal cell deficiency, dry eye, glaucoma, familial exudative vitreoretinopathy (FEVR), Norrie disease, Coats disease, retinopathy of prematurity, macular telangiectasia, retinal vein occlusion, Sjögren's syndrome, sensorineural hearing loss, conductive hearing loss, schizophrenia, Parkinson's disease, polycystic kidney disease, focal segmental glomerulosclerosis, chronic obstructive pulmonary disease, idiopathic pulmonary fibrosis, short bowel syndrome, melanoma, pancreatic cancer, prostate cancer, colon cancer, leukemia, septic shock, ischemia, inflammation, pulmonary hypertension, congestive heart failure, cardiovascular disease, dermatological disease, cancer or GM2 gangliosidosis.

N-desmethyl ruboxistaurin, or its pharmaceutically acceptable salt, or a pharmaceutical composition thereof, can be administered in combination with lithium for bipolar disorder or other conditions where inhibition of GSK3, protein kinase C, or both, is useful. Bipolar disorder co-treatments, other than lithium, can include valproic acid, lamotrigine, quetiapine, olanzapine, risperidone, aripiprazole, lurasidone, lumateperone, cariprazine, asenapine, and carbamazepine.

Lithium can be administered at a sub-effective dose based on monotherapy, and N-desmethyl ruboxistaurin can be administered at a sub-effective dose based on monotherapy. The sub-effective dose of lithium can be a dose that reduces, or does not cause, kidney damage.

The subject can be non-responsive to lithium, or lithium responsive.

Another aspect of the invention is directed to a method of establishing a diagnosis of bipolar disorder or other condition where GSK3 inhibition is clinically useful, by administering to a subject to be evaluated a therapeutically effective dose of N-desmethyl ruboxistaurin or a pharmaceutically acceptable salt thereof, or a pharmaceutical composition thereof, and evaluating the subject's clinical response.

A further aspect of the invention is directed to a method of establishing an appropriate therapeutic dose of N-desmethyl ruboxistaurin in a subject, by administering increasing doses of N-desmethyl ruboxistaurin to the subject and assessing response using GSK3 imaging or GSK3 serology.

Yet another aspect of the invention is directed to a method of treating a subject with Alzheimer's disease, bipolar disorder, or depression, who shows evidence of elevated GSK3, by administering to the subject a therapeutically effective dose of N-desmethyl ruboxistaurin or a pharmaceutically acceptable salt thereof, or a pharmaceutical composition thereof, and evaluating and monitoring the subject using positron emission tomography (PET) or serology.

Still another aspect of the invention is directed to a method of establishing a diagnosis of bipolar disorder or other condition where GSK3 inhibition is clinically useful, by administering to a subject to be evaluated a therapeutically effective dose of N-desmethyl ruboxistaurin or a pharmaceutically acceptable salt thereof, or a pharmaceutical composition thereof, with a therapeutically effective dose of lithium, and evaluating the subject's clinical response. The dose of both N-desmethyl ruboxistaurin and lithium can be sub-effective based on monotherapy.

An additional aspect of the invention is directed to a method of treating a subject with Alzheimer's disease who has evidence of elevated GSK3 activity, by administering to the subject a therapeutically effective dose of N-desmethyl ruboxistaurin or a pharmaceutically acceptable salt thereof, or a pharmaceutical composition thereof, and a therapeutically effective dose of lithium, and monitoring the subject using positron emission topography (PET). The dose of both N-desmethyl ruboxistaurin and lithium can be sub-effective based on monotherapy.

Another aspect of the invention is directed to a method of establishing an appropriate therapeutic dose of N-desmethyl ruboxistaurin in a subject, by administering increasing doses of N-desmethyl ruboxistaurin and lithium to the subject and assessing response using positron emission topography (PET).

### BRIEF DESCRIPTION OF THE DRAWINGS

FIG. 1 shows the QT interval on an electrocardiogram (ECG) tracing, which describes that portion of the cardiac contraction cycle that starts with contraction of the left ventricle and ends with the relaxation of the left ventricle.

FIG. 2 shows a diagram of the therapeutic window for a drug on a graph of % maximum effect versus drug concentration.

FIG. 3 displays the chemical structure of ruboxistaurin.

FIG. 4 displays the chemical structure of N-desmethyl ruboxistaurin.

FIG. 5 shows a graph of the mean plasma concentration versus time profile following single-dose administration of 32 mg ruboxistaurin in healthy subjects. Results in the insert are in the fed state.

FIG. 6 shows a synthetic scheme for N-desmethyl ruboxistaurin.

FIG. 7 shows the ability of ruboxistaurin and N-desmethyl ruboxistaurin to inhibit GSK3β and GSK3α at various concentrations.

FIG. 8 displays the stability of ruboxistaurin and N-desmethyl ruboxistaurin in human liver microsomes.

FIG. 9 shows the ability of N-desmethyl ruboxistaurin to provide a pharmacological effect similar to lithium in rats, reducing positive ultrasonic vocalizations that are induced by dextroamphetamine.

### DETAILED DESCRIPTION

Unless otherwise defined, all terms used in disclosing the invention, including technical and scientific terms, have the meaning as commonly understood by one of ordinary skill in the art to which this invention belongs.

As disclosed herein, several ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges can independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention. The term “about” generally includes up to plus or minus 10% of the indicated number. For example, “about 10%” can indicate a range of 9% to 11%, and “about 20” can mean from 18 to 22. Preferably “about” includes up to plus or minus 6% of the indicated value. Alternatively, “about” includes up to plus or minus 5% of the indicated value. Other meanings of “about” may be apparent from the context, such as rounding off, so, for example “about 1” can also mean from 0.5 to 1.4.

The term “pharmaceutically acceptable salt” of a compound means a salt that is pharmaceutically acceptable and that possesses the desired pharmacological activity of the parent compound. It is understood that the pharmaceutically acceptable salts are non-toxic. Such salts include acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. Additional information on suitable pharmaceutically acceptable salts can be found in Remington's Pharmaceutical Sciences, 17th ed., Mack Publishing Company, Easton, PA, 1985, which is incorporated herein by reference.

As used herein, the term “therapeutically effective amount” means an amount of compound of the present invention which is capable of alleviating the symptoms of the various pathological conditions herein described. The specific dose of a compound administered according to this invention will, of course, be determined by the particular circumstances surrounding the case including, for example, the compound(s) administered, the route of administration, the state of being of the patient, and the pathological condition being treated. Dosing can be once per day, or administered in multiple sub-doses per day, e.g., two, three, or more doses per day.

The effective dose of N-desmethyl ruboxistaurin, or pharmaceutically acceptable salt thereof, is about 32 to about 320 mg once daily, or about 16 to about 160 mg twice daily for monotherapy. A pharmaceutical composition of N-desmethyl ruboxistaurin, or pharmaceutically acceptable salt thereof, further comprises at least one pharmaceutically acceptable adjuvant or excipient.

For combination therapy, a sub-effective dose of N-desmethyl ruboxistaurin, or pharmaceutically acceptable salt thereof, is about 8 to about 32 mg once daily, or about 4 to about 16 mg twice daily. When N-desmethyl ruboxistaurin is combined with lithium, a sub-effective dose of lithium can be about 60 mg to about 600 mg once daily, or about 30 mg to about 300 mg twice daily. This sub-effective dose of lithium can spare the kidney damage typically caused by lithium treatment. The once daily effective dose of N-desmethyl ruboxistaurin, or pharmaceutically acceptable salt thereof, can be about 32 or about 64 or about 96 or about 128 or about 160 or about 192 or about 224 or about 256 or about 288 or about 320 mg. The twice daily effective dose of N-desmethyl ruboxistaurin, or pharmaceutically acceptable salt thereof, can be about 16 or about 32 or about 48 or about 64 or about 80 or about 96 or about 112 or about 128 or about 144 or about 160 mg. The once daily sub effective dose of N-desmethyl ruboxistaurin, or pharmaceutically acceptable salt thereof, can be about 8 or about 16 or about 24 or about 32 mg. The twice daily sub effective dose of N-desmethyl ruboxistaurin, or pharmaceutically acceptable salt thereof, can be about 4 or about 8 or about 12 or about 16 mg.

Ruboxistaurin has been investigated in several clinical trials for the treatment of diabetes mellitus and its complications, including diabetic retinopathy, diabetic neuropathy, and diabetic nephropathy. See A. Girach, US Patent Publication No. 2008/0096923, incorporated herein by reference in its entirety. Its safety has been described as excellent, with a lower incidence of serious adverse events than placebo. Further development of ruboxistaurin has been encouraged because of its clinical safety profile, and because of growing interest in the use of treatments that can inhibit GSK3. Furthermore, ruboxistaurin is currently proposed to enter clinical trials soon for the treatment of GM2 gangliosidosis.

However, careful review of ruboxistaurin clinical and preclinical data has identified its potential to prolong the QT interval in human subjects, and this can increase the risk of dangerous cardiac arrhythmias, particularly in the case of an accidental or intentional overdose of ruboxistaurin, or when administered with other drugs that prolong the QT interval, or when very high ruboxistaurin levels are induced by co-administration of a drug inhibiting CYP3A4.

Evidence for these potential risks lie in the Withdrawal Assessment Report prepared by the European Medicines Agency (EMA). A Marketing Authorization Application for ruboxistaurin had been submitted to the European Medicines Agency, and the application was withdrawn, prompting the report. The Withdrawal Assessment Report showed that ruboxistaurin inhibits hERG (a potassium ion channel known for its contribution to electrical activity of the heart). This preclinical assessment is commonly used to identify compounds with a potential risk for QT prolongation.

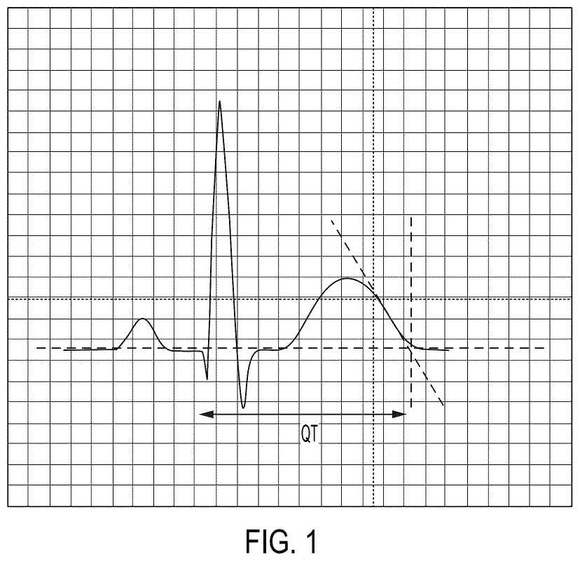

The QT interval is part of an electrocardiogram (ECG) tracing, describing that portion of the cardiac contraction cycle that starts with contraction of the left ventricle (the letter “Q” representing initiation of a “QRS” complex in the waveform) and ends with the relaxation of the left ventricle (termination of the “T” wave). A QT interval should be generally 425 msec or less. If the QT interval is too long, the heart does not finish relaxing before the next electrical signal is initiated, and this can cause dangerous arrhythmias. See FIG. 1.

In the hERG assay, ruboxistaurin blocked the hERG channel with an IC₅₀ value of 35.6 nM. This led to a predicted increase of 5 msec in the QT interval. Further assessment of cardiac safety in dogs did not show an issue, but there were concerns with the results of a “thorough QT” study performed in human subjects with ruboxistaurin. This type of study is done with numerous ECG tracings at different time intervals after exposure to study drug.

Ruboxistaurin prolonged the QT interval by 6 msec in the thorough human QT study. Moreover, there were several patients identified in the study whose QT interval was prolonged by more than 10 msec. This potential safety concern was the basis of a major objection by the European Medicines Agency.

A positive hERG assay predicts QT prolongation, and QT prolongation has been associated with life-threatening cardiac arrhythmias, including Torsades de Pointes. These studies alone are not perfect predictors of cardiac risk. However, avoidance of QT prolongation is considered clinically desirable. Pharmaceutical companies have at times halted development of products that prolong the QT interval by more than 5 msec.

Even though a drug with QT prolongation can be approved, the prescribing instructions can come with safety warnings like those of ziprasidone, whose FDA label states, “When deciding among the alternative treatments available for schizophrenia, the prescriber should consider the finding of ziprasidone's greater capacity to prolong the QT/QTc interval compared to several other antipsychotic drugs (see WARNINGS).” While Torsades de Pointes is not in the ziprasidone FDA label, cardiologists have preferred the use of other medications within the class, precisely because of concerns around QT prolongation.

QT prolongation raises a greater concern for potential cardiac arrhythmias when high doses are administered, in the case of concomitant use of other drugs that prolong the QT interval, in the case of accidental or intentional overdoses, and in cases where plasma exposure to a drug is high, such as where the metabolism of a drug is inhibited by interactions with other drugs. All these situations apply to ruboxistaurin.

High doses of ruboxistaurin are currently being investigated for treatment of congestive heart failure. While 32 mg of ruboxistaurin was used extensively in the development of ruboxistaurin for the treatment of diabetic retinopathy, doses of up to 256 mg are being explored for the treatment of heart failure. One of the primary outcome measures in the heart failure study is the proportion of patients with elongation of the QT interval.

The pharmacokinetics of a compound are also important in considering the potential for QT prolongation. The potential for arrhythmias relates to the maximum plasma concentration of the compound. This has been observed in ECG studies where the maximum prolongation of the QT interval is shown at the time of maximum concentration of study drug.

A high peak concentration causing a serious prolongation of the QT interval might not be present in most patients for most of the time, but it can become a problem when there is an accidental or intentional overdose. This principle is evident in guidance for the use of tricyclic antidepressants. Physicians are taught to monitor the ECG of a patient during treatment with tricyclic antidepressants, which prolong the QT interval. Importantly, physicians are also taught to reduce the potential for a dangerous overdose and cardiac arrhythmias by prescribing only small amounts at a time. In principle, an overdose of 30 tablets is safer than an overdose of 100 tablets.

In normal use, the peak concentration of a drug can be reduced (in relation to the trough concentration of the drug) when the half-life is longer. For example, a drug with a half-life of 24 hours can be given once a day with a peak drug concentration that is only about 2 times the trough concentration. In contrast, a drug with a half-life of 6 hours, if given once a day, may have a peak concentration approximately 16 times the trough concentration.

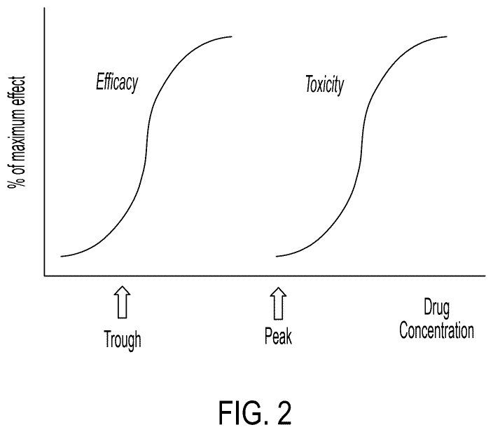

A lower peak concentration (in relation to the trough concentration) is generally desirable as a principle of drug development because it keeps drug levels within the therapeutic window. There is a dose response for efficacy and a dose response for toxicity. A lower peak/trough ratio can help keep a drug concentration at levels that provide efficacy without being high enough to cause toxicity. See FIG. 2.

Ruboxistaurin has a high peak concentration of about 90 nmol/L compared to its trough concentration of about 5 nmol/L (a peak trough ratio of about 18, consistent with a half-life of less than 6 hours). This raises the risk not just for QT prolongation but also other toxicities, of which worsening glucose control and elevations in creatine kinase were of concern for the European Medicines Agency.

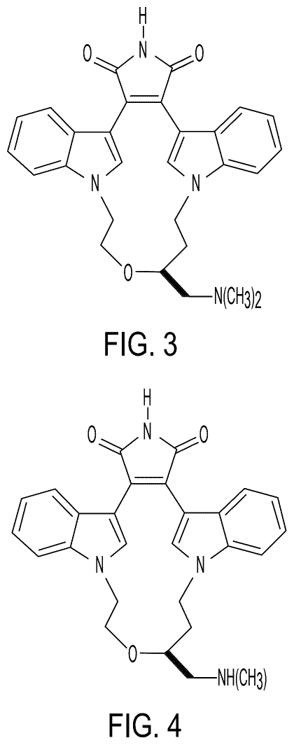

A third consideration affecting the peak concentration of a drug is potential interactions with other drugs. Ruboxistaurin is metabolized by CYP3A4, which converts ruboxistaurin to N-desmethyl ruboxistaurin. See FIGS. 3 and 4. The peak concentration of ruboxistaurin might therefore be elevated in the presence of a CYP3A4 inhibitor.

These considerations are especially relevant in conditions where patients may take one or more medicines prolonging the QT interval. Medications that prolong the QT interval include antipsychotics (haloperidol, ziprasidone, quetiapine, thioridazine, olanzapine, risperidone), antiarrhythmics (amiodarone, sotalol, dofetilide, procainamide, quinidine, flecainide), antibiotics (macrolides, fluoroquinolones), antidepressants (amitriptyline, imipramine, citalopram) and others (methadone, sumatriptan, ondansetron, cisapride).

Drugs that prolong the QT interval are frequently prescribed in the case of bipolar disorder, depression, and/or schizophrenia. Patients with these conditions may be prescribed antidepressants. And they may be prescribed atypical antipsychotics, which are indicated for the treatment of bipolar disorder, depression and schizophrenia.

QT prolongation is also a concern because patients with bipolar disorder and depression are at increased risk of attempting suicide, which may involve drug overdoses. Further, confusion or delusion in patients with mental illness may lead to accidental medication overdose.

Frequent prescription of drugs prolonging the QT interval also occurs in the case of Alzheimer's disease. Depression or agitation may be present (behavioral complications of dementia), and these conditions may be treated with antidepressants or atypical antipsychotics, or both simultaneously. Patients with Alzheimer's disease are generally older, increasing the risk that they are prescribed antibiotics and other medications. Further, confusion or delusion in patients with Alzheimer's disease may lead to accidental medication overdose.

Ruboxistaurin is metabolized by CYP3A4, whose inhibitors include grapefruit juice, itraconazole, voriconazole, ketoconazole, ritonavir, boceprevir, danoprevir, telaprevir, saquinavir, azamulin, erythromycin, troleandomycin, telithromycin, verapamil, diltiazem, ciprofloxacin, cyclosporine, imatinib, cimetidine, ranitidine, and the antidepressants nefazodone and fluvoxamine. The use of these medications has the potential to increase peak concentrations of ruboxistaurin and worsen prolongation of the QT interval.

For these reasons, the use of ruboxistaurin is a particular concern in treating neuropsychiatric disorders, including bipolar disorder, depression, and Alzheimer's disease, all of which are indications where new medications are needed and research on the use of GSK3 inhibitors and ruboxistaurin has been encouraged. The use of ruboxistaurin is also a concern in treating elderly subjects, as the elderly often take multiple medications and are at higher risk of adverse drug interactions.

Further, the half-life of ruboxistaurin is sub-optimal for its combination with lithium. The elimination half-life of lithium is about 18 hours. A combination product of two medicines should have both products with levels within the therapeutic window throughout the dosing interval. Combining two products, one with a short half-life and one with a long-half life, leads to unnecessarily high levels of the short-acting drug soon after administration, with the potentially low levels for that same component later in the dosing interval.

A new drug can be an important alternative to ruboxistaurin if it has similar or better potency against protein kinase C or GSK3 and at the same time has at least one or more of the following features: a similar or lower inhibition of hERG, a longer half-life that allows a lower peak concentration during the dosing interval (better pharmacokinetics), and a lower potential for drug-drug interactions. An aspect of this invention is directed to using N-desmethyl ruboxistaurin, which has these features, in situations where ruboxistaurin is clinically useful.

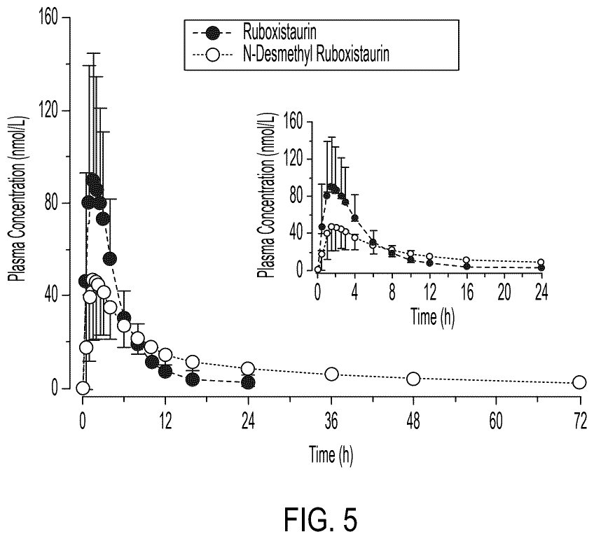

In the hERG assay, the ability for N-desmethyl ruboxistaurin to block the hERG channel was reported to be somewhat lower than that of ruboxistaurin, requiring higher concentrations, with an IC50 of 62.6 nM for N-desmethyl ruboxistaurin vs. 35.6 nM for ruboxistaurin. The half-life (t½) of N-desmethyl ruboxistaurin was also reported to be longer than that of ruboxistaurin, 23.9 hours compared to 5.25 hours (FIG. 5). Inhibitors of CYP3A4 can elevate plasma levels of ruboxistaurin, but they have not been reported to elevate plasma levels of N-desmethyl ruboxistaurin to the same extent, and the long half-life of N-desmethyl suggests another pathway of metabolism and elimination, hydroxylation. Therefore N-desmethyl ruboxistaurin may have a somewhat lower potential to inhibit hERG, has a better pharmacokinetic profile, can reduce interactions with CYP3A4 inhibitors, and has a half-life more suitable for a combination with lithium.

The difference in IC₅₀ for hERG is reported to be about two-fold for N-desmethyl ruboxistaurin vs. ruboxistaurin, and the peak plasma levels are reported to be only half that of N-desmethyl ruboxistaurin vs. ruboxistaurin, so the combination of these features can reduce the potential for N-desmethyl ruboxistaurin to prolong the QT interval or provoke other toxicities to a level only about % that of ruboxistaurin. And in the presence of a drug that inhibits CYP3A4, the difference becomes even greater.

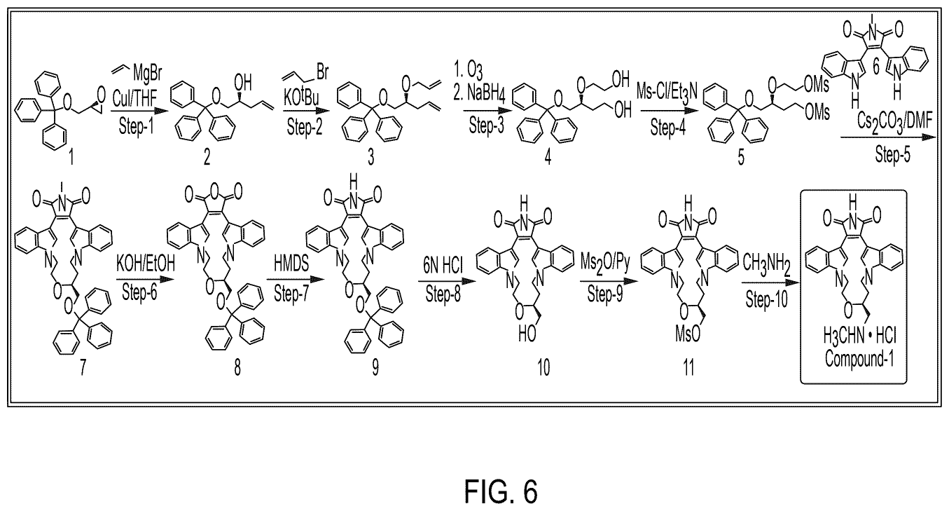

Preparation of N-desmethyl ruboxistaurin (Compound-1) generally follows the methods illustrated in FIG. 6. As shown, starting material 1 is reacted with vinyl Grignard in the presence of copper iodide giving alcohol intermediate 2. One of ordinary skill in the art will recognize that alternatives to vinyl Grignard are useful in effecting the same transformation. Such alternatives include, but are not limited to, vinyl zinc reagents, vinyl cuprate reagents and vinyl lithium reagents. One of ordinary skill in the art will recognize that alternatives to copper iodide are useful in facilitating conversion of intermediate 1 to intermediate 2. Such alternatives include, but are not limited to, alternate Lewis acid reagents and chelating agents such as crown ethers.

Following isolation of intermediate 2, FIG. 6 illustrates conversion to intermediate 3 on reaction with allyl bromide. One of ordinary skill in the art will recognize that alternate allylating agents are useful in the allylation of intermediate 2 to intermediate 3. Such allylating agents generally employ alternates to the bromide leaving group and include, but are not limited to, allyl chloride, allyl iodide and allyl mesylate. One of ordinary skill in the art will also recognize that alternates to the illustrated potassium tert-butoxide base are useful in effecting the reaction of intermediate 2 with an allylating agent. Such bases include, but are not limited to, hydride reagents, carbonate reagents, bicarbonate reagents, lithium diisopropylamide, sodium hexamethyl disilazide and the like.

FIG. 6 further illustrates the 2-step conversion of intermediate 3 to intermediate 4. As shown, the first step is an ozonolysis reaction leading to the cleavage of a bis-olefin to a bis-aldehyde and the second step is a sodium borohydride reduction of a bis-aldehyde to a bis-alcohol. One of ordinary skill in the art will recognize that ozonolysis is only one of many reactions or reaction combinations suitable for cleavage of an olefin to an aldehyde. Such conversions include, but are not limited to, dihydroxylation of an olefin followed by cleavage of the resulting diol to an aldehyde. Suitable reagents for the dihydyroxylation of an olefin include, but are not limited to, osmium tetroxide and the like. Suitable reagents for the cleavage of a diol to an aldehyde include, but are not limited to, sodium periodate, lead tetraacetate and the like. One of ordinary skill in the art will further recognize that alternates to the sodium borohydride reducing agents are suitable for the reduction of aldehydes to alcohols. Such reagents include, but are not limited to, lithium aluminum hydride, diisopropyl aluminum hydride, lithium borohydride, borane and the like. One of ordinary skill in the art will additionally recognize that alternatives to boron and aluminum-based reducing agents are also useful for the reduction of aldehydes to alcohols. Such alternatives include, but are not limited to, samarium iodide and triethylsilane.

As illustrated in FIG. 6, the diol of intermediate 4 is converted to the bis-mesylate intermediate 5 on reaction with methanesulfonyl chloride and triethylamine. One of ordinary skill in the art will recognize that mesylates, as leaving groups, are generally useful as are common alternative leaving groups including, but not limited to, chlorides, bromides, iodides, tosylates and the like. One of ordinary skill in the art will also recognize that alternatives to triethylamine are useful in the conversion of alcohols to mesylates with such alternatives including, but not being limited to, diisopropyl ethylamine, pyridine, carbonate reagents, bicarbonate reagents and the like.

The reaction of the bis-mesylate intermediate 5 with the bis-indolyl maleimide intermediate 6 forming intermediate 7 is illustrated in FIG. 6 using cesium carbonate as the base. One of ordinary skill in the art will recognize that alternative bases are useful for effecting the illustrated reaction to intermediate 7. Such bases include, but are not limited to, hydrides, alkoxides, carbonates, bicarbonates and the like.

The process of converting the methyl maleimide to its demethylated version involves initial hydrolysis of intermediate 7 to the maleic anhydride intermediate 8. As illustrated in FIG. 6, this transformation is effected using potassium hydroxide in ethanol. One of ordinary skill in the art will recognize that conversion of intermediate 7 to intermediate 8 can employ alternates to potassium hydroxide include, but are not limited to, sodium hydroxide and lithium hydroxide. Furthermore, one of ordinary skill in the art will recognize that ethanol can be exchanged for any protic solvent including, but not limited to, methanol and water.

According to FIG. 6, maleic anhydride intermediate 8 is to the corresponding maleimide intermediate 9 is accomplished on reaction of intermediate 8 with hexamethyldisilazane. One of ordinary skill in the art will recognize that maleic anhydrides can be converted to maleimides using alternate reagents including, but not limited to, ammonia, sodamide and the like.

With the maleimide established, FIG. 6 illustrates cleavage of the trityl protecting group from intermediate 9 to alcohol intermediate 10. While FIG. 6 highlights hydrochloric acid as the reagent effecting trityl cleavage, one of ordinary skill in the art will recognize that alternate acids can be used. Said alternates include, but are not limited to, hydrobromic acid, trifluoroacetic acid, acetic acid and the like.

As illustrated in FIG. 6, the alcohol of intermediate 10 is converted to the mesylate intermediate 11 on reaction with methanesulfonyl chloride and pyridine. One of ordinary skill in the art will recognize that mesylates, as leaving groups, are generally useful as are common alternative leaving groups including, but not limited to, chlorides, bromides, iodides, tosylates and the like. One of ordinary skill in the art will also recognize that alternatives to triethylamine are useful in the conversion of alcohols to mesylates with such alternatives including, but not being limited to, diisopropyl ethylamine, pyridine, carbonate reagents, bicarbonate reagents and the like.

In the final stage of the synthesis, FIG. 6 illustrates conversion of intermediate 11 to Compound-1 on reaction with methylamine. While not illustrated in FIG. 6, the methylamine is further converted to its corresponding hydrochloride salt. One of ordinary skill in the art will understand that alternate strategies for conversion of compounds such as intermediate 11 to structures such Compound-1 exist. Such strategies are generally recognizable by one skilled in the art and said strategies are generally supported by resources such as Comprehensive Organic Transformations (Larock, Wiley).

One of ordinary skill in the art will recognize that there are many additional reactions, in addition to those described above and illustrated in FIG. 6, that are useful for the preparation of N-desmethyl ruboxistaurin (Compound-1). Suitable reactions are readily identified by one skilled in the art and are available in resources such as Comprehensive Organic Transformations (Larock, Wiley). Strategies for introduction and cleavage of protecting groups are readily identified by one skilled in the art and are available in resources such as Protective Groups in Organic Synthesis (Greene and Wutz, Wiley). One of ordinary skill in the art will further recognize that, generally applicable to FIG. 6 and to any and all alternates and variations to the route described in FIG. 6, alternate combinations of reagents, solvents, temperature conditions and reaction times will provide similar chemical outcomes enabling the preparation of Compound-1.

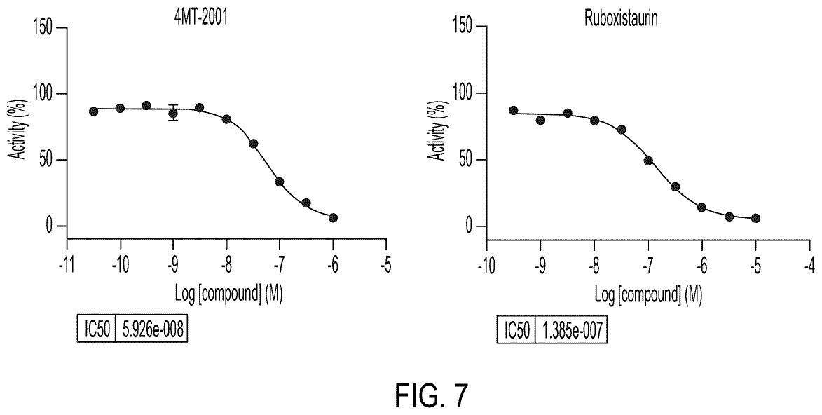

The ability of N-desmethyl ruboxistaurin to inhibit GSK3β (a specific form of GSK linked to bipolar disorder and other neuropsychiatric disorders) was previously unknown. Surprisingly, N-desmethyl ruboxistaurin was found to be approximately twice as potent as ruboxistaurin in its inhibition of GSK3β (FIG. 7). This greater potency provides an ability to use N-desmethyl ruboxistaurin at lower doses than ruboxistaurin.

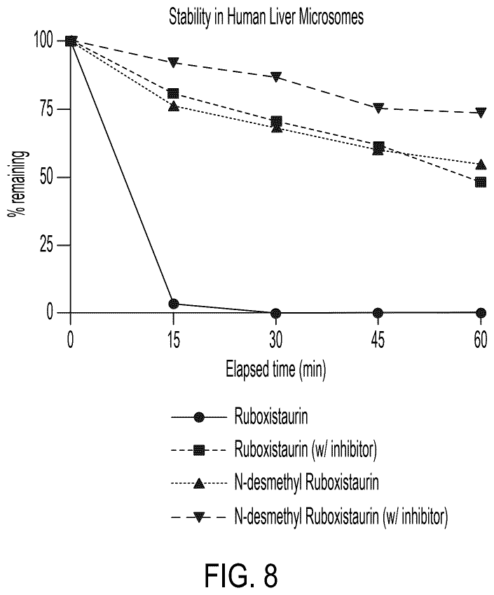

N-desmethyl ruboxistaurin was found to have greater stability compared to ruboxistaurin in human liver microsomes. 76.38% of N-desmethyl ruboxistaurin was found to remain after 15 minutes in the liver respective to 3.51% of ruboxistaurin remaining (FIG. 8). In this study, the CYP3A4 inhibitor troleandomycin increased ruboxistaurin levels at 15 minutes by 23-fold, while N-desmethyl ruboxistaurin levels were only increased 1.2-fold. This indicates that CYP3A4 affects the metabolism of N-desmethyl ruboxistaurin less than that of ruboxistaurin (Table 1).

TABLE 1
Metabolic stability in pooled human liver
microsomes with and without inhibitor

| | CYP | Remaining percentage (%) | | | | |
|---|---|---|---|---|---|---|
| Compound | isoform | 0 min | 15 min | 30 min | 45 min | 60 min |
| Ruboxistaurin | With | 100 | 80.77 | 70.58 | 61.54 | 48.17 |
| | inhibitor | | | | | |
| | Without | 100 | 3.51 | 0.00 | 0.00 | 0.00 |
| | inhibitor | | | | | |
| N-Desmethyl | With | 100 | 91.99 | 86.56 | 75.23 | 73.47 |
| Ruboxistaurin | inhibitor | | | | | |
| | Without | 100 | 76.38 | 68.32 | 59.98 | 55.03 |
| | inhibitor | | | | | |

The ability of N-desmethyl ruboxistaurin to cross the blood-brain barrier was previously unknown. A pharmacokinetic study in rats showed brain penetration of N-desmethyl ruboxistaurin, with a brain/plasma ratio of 1.18 after 4 hours, supporting the potential for therapeutic use of N-desmethyl ruboxistaurin to treat conditions of the central nervous system (Table 2).

TABLE 2
Concentrations of N-desmethyl ruboxistaurin
in plasma and brain of rats

| Time | Mean plasma | Mean brain | |
|---|---|---|---|
| (hr) | (ng/mL) | (ng/g) | Mean brain/plasma |
| 0.25 | 73.3 | 21.0 | 0.294 |
| 1 | 43.5 | 19.5 | 0.452 |
| 4 | 11.8 | 13.3 | 1.18 |

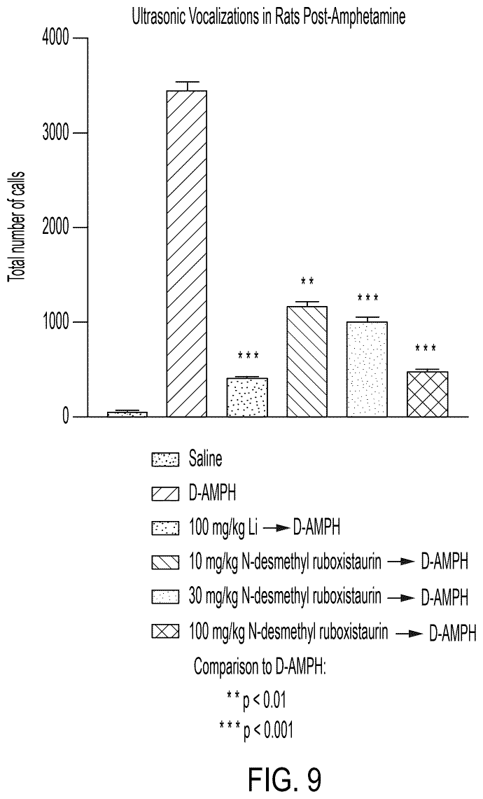

The ability of N-desmethyl ruboxistaurin to have a pharmacological effect similar to lithium was previously unknown. N-desmethyl ruboxistaurin was able to reduce ultrasonic appetitive vocalizations in rats treated with dextroamphetamine, and the magnitude of effect was similar to that of lithium (FIG. 9). This supports the potential for N-desmethyl ruboxistaurin to provide clinical benefits similar to lithium.

As an alternative to ruboxistaurin, N-desmethyl ruboxistaurin can be administered to a subject who has never used ruboxistaurin. As an alternative to ruboxistaurin, N-desmethyl ruboxistaurin can be administered to a subject who has experienced adverse effects of ruboxistaurin, has a prolonged QT interval, has been shown to have high drug levels of ruboxistaurin, or has the potential to receive drugs that may interfere with the metabolism of ruboxistaurin, or where higher doses of ruboxistaurin might be needed and may cause a concern of adverse effects, QT prolongation, or adverse drug interactions.

The risk of QT prolongation and other potential toxicities can be further reduced by administering N-desmethyl ruboxistaurin together with lithium. Both N-desmethyl ruboxistaurin and lithium inhibit GSK3, and it has been shown that lithium with another GSK3 inhibitor has synergy in treating bipolar disorder in an animal model. A desired amount of GSK3 inhibition can be achieved with a lower concentration of N-desmethyl ruboxistaurin if it is administered in conjunction with lithium. Thus, N-desmethyl ruboxistaurin, or its pharmaceutically acceptable salt, can be administered in combination with lithium for bipolar disorder, or other conditions where inhibition of GSK3, protein kinase C, or both, is useful.

While this combination can lower the dose of N-desmethyl ruboxistaurin, it can also lower the dose of lithium required for the treatment of bipolar disorder, Alzheimer's disease, and other conditions where GSK3 inhibition by lithium is clinically desirable, and thereby improve safety. The dose of N-desmethyl ruboxistaurin in the combination can be a dose lower than what would be needed as monotherapy (a sub-effective dose), and the dose of lithium in the combination can be a dose lower than what would be needed as monotherapy (a sub-effective dose). The combination can be used to provide efficacy in subjects non-responsive to lithium or intolerant of lithium at standard doses. N-desmethyl ruboxistaurin can be used to provide additional efficacy in subjects who have only a partial response to lithium, as an alternative to higher lithium doses.

Furthermore, ruboxistaurin has been proposed in combination with valproic acid, lamotrigine, carbamazepine, gabapentin, and topiramate for the treatment of a neurological disease and/or a psychiatric disorder. With superior pharmacokinetics and a lower potential for QT prolongation, N-desmethyl ruboxistaurin can be used as an alternative to ruboxistaurin in combination with valproic acid, lamotrigine, quetiapine, olanzapine, risperidone, aripiprazole, lurasidone, lumateperone, cariprazine, asenapine, and carbamazepine.

Antipsychotics not known to prolong the QT interval, which include olanzapine, risperidone, aripiprazole, lumateperone, xanomeline-trospium, iloperidone and lurasidone, have also been used for the treatment of neurological disease and/or psychiatric disorders including bipolar disorder, depression, Parkinson's disease and schizophrenia. N-desmethyl ruboxistaurin can be combined with these antipsychotics to treat these conditions.

Furthermore, a response to either N-desmethyl ruboxistaurin or the combination of N-desmethyl ruboxistaurin with lithium can serve to establish a diagnosis of bipolar disorder and other conditions where GSK3 inhibition is clinically useful. In Alzheimer's disease, positron emission tomography (PET) of GSK3β activity is being developed as a diagnostic. N-desmethyl ruboxistaurin, alone or in combination with lithium, may be administered to subjects with excess GSK3β activity on PET in order to treat Alzheimer's disease, and a reduction in GSK3β activity on PET after administration of N-desmethyl ruboxistaurin can support its use (alone or in combination with lithium) as an appropriate therapy administered at a suitable dose.

The presently disclosed compositions and treatment methods are relevant wherever ruboxistaurin may be clinically useful, including psychiatric and neurological disorders, such as bipolar disorder, depression, Alzheimer's disease, frontotemporal dementia, behavioral complications of dementia, autism spectrum disorder, Fragile X syndrome, Pitt Hopkins syndrome, Rett syndrome, traumatic brain injury, stroke, acute spinal cord injury, schizophrenia, Parkinson's disease, amyotrophic lateral sclerosis (ALS), multiple sclerosis (MS), neurofibromatosis type 1, neuronal ceroid lipofuscinosis, chronic pain, neuropathic pain, chemotherapy-induced neuropathy, and chemotherapy-induced cognitive impairment.

Ruboxistaurin and N-desmethyl ruboxistaurin are reported to be equipotent with respect to inhibiting protein kinase C, and therefore these compositions and treatment methods are relevant to indications where ruboxistaurin may be applied as a protein kinase C inhibitor, including diabetes mellitus, diabetic nephropathy, diabetic neuropathy, diabetic retinopathy, ischemia, inflammation, cardiovascular disease, pulmonary hypertension, congestive heart failure, dermatological disease, cancer and GM2 gangliosidosis.

These composition and treatment methods are also relevant to conditions where GSK3 inhibition and or enhancement of WNT signaling have been proposed, including alopecia, osteoarthritis, osteoporosis, alcoholic hepatitis, inflammatory bowel disease, wet age-related macular degeneration, dry age-related macular degeneration, diabetic macular edema, Fuch's dystrophy, limbal cell deficiency, dry eye, glaucoma, familial exudative vitreoretinopathy (FEVR), Norrie disease, Coats disease, retinopathy of prematurity, macular telangiectasia, retinal vein occlusion, Sjögren's syndrome, sensorineural hearing loss, conductive hearing loss, schizophrenia, Parkinson's disease, polycystic kidney disease, focal segmental glomerulosclerosis, chronic obstructive pulmonary disease, idiopathic pulmonary fibrosis, short bowel syndrome, melanoma, pancreatic cancer, prostate cancer, colon cancer, leukemia, septic shock, and ischemia/reperfusion injury. Such compositions and methods are also relevant to GM2 gangliosidosis, where the use of ruboxistaurin has been proposed.

The presently disclosed compositions and treatment methods also have use in veterinary applications for improving the health and well-being of livestock and companion animals by treating any of the foregoing indications that occur in animals.

### EXAMPLES

### Materials and Methods

### Example 1. Compound Synthesis: N-Desmethyl Ruboxistaurin was Synthesized According to the Following Procedures

### Step-1: Synthesis of (S)-1-(trityloxy) pent-4-en-2-ol (2)

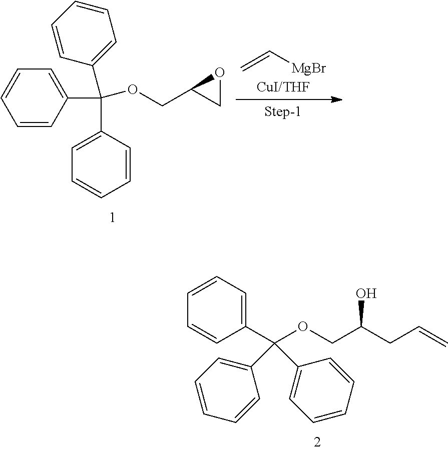

To a solution of vinyl magnesium bromide (1M in THF, 840 mL, 0.84 mol) was added copper iodide (4.5 g, 23.62 mmol) at −40° C., under nitrogen atmosphere. After stirring for 20 min at −40° C., Compound-1 (150 g, 0.46 mmol), dissolved in dry THF (750 mL) was added dropwise into the reaction mixture, and the resulting reaction mixture was stirred at −40° C. for 2 h. After completion of the reaction, (monitored by TLC), sat. ammonium chloride (1000 mL) was added. Warmed the reaction to RT, while stirring and extracted with ethyl acetate (1000 mL). Separated the organic layer and washed with aq. Ammonia (250 mL). Separated the organic layer, dried over sodium sulphate, filtered and evaporated under vacuum to obtain Compound-2 as a dark brown sticky material (166 grams, 100% crude yield). ¹H NMR (400 MHz, CDCl₃): δ 7.45-7.42 (m, 6H), 7.32-7.28 (m, 6H), 7.26-7.22 (m, 3H), 5.74-5.71 (m, 1H), 5.09-5.02 (m, 2H), 3.85-3.82 (m, 1H), 3.18 (dd, J=9.6 Hz, J=4.0 Hz, 1H), 3.09 (dd, J=9.2 Hz, J=6.8 Hz, 1H), 2.27-2.22 (m, 3H).

### Step-2: Synthesis of (S)-(((2-(allyloxy) pent-4-en-1-yl) oxy) methane trityl) tribenzene (3)

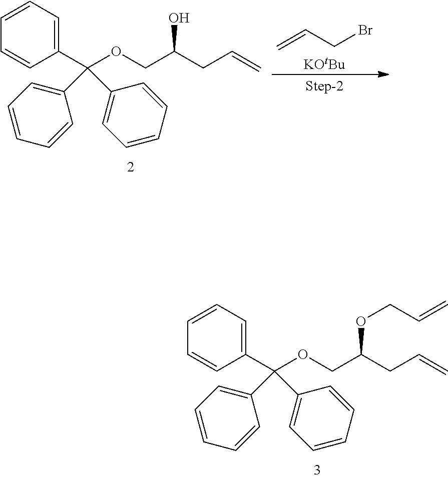

To a stirred solution of compound-2 (165 g, 0.48 mol) in dry THF (1500 mL) was added potassium tert-butoxide (70.0 g, 0.62 mmol) portion-wise, under nitrogen atmosphere. The resulting reaction contents were heated to 45° C. and stirred for 2 h, then cooled to RT, followed by the addition of allyl bromide (145.5 g, 1.22 mol) at RT and continued the stirring for 1 h at RT. After completion of the reaction (monitored by TLC), added sat. ammonium chloride (1500 mL) into the reaction and extracted with ethyl acetate (1500 mL). Separated the organic layer, dried over sodium sulphate, filtered and evaporated under vacuum to give the crude compound-3, which was further purified by silica gel column chromatography (100-200 mesh), eluting with 0.5-1% ethyl acetate in hexane. The pure fractions were collected and evaporated under reduced pressure to afford the desired compound-3 as a pale yellow semi-solid (106 grams, 58% yield). ¹H NMR (400 MHz, CDCl₃): δ 7.48-7.44 (m, 6H), 7.31-7.26 (m, 6H), 7.25-7.20 (m, 3H), 5.95-5.88 (m, 1H), 5.75-5.70 (m, 1H), 5.27 (dd, J=17.2 Hz, J=2.0 Hz, 1H), 5.15 (dd, J=10.4 Hz, J=2.0 Hz, 1H), 5.03 (dd, J=17.2 Hz, J=2.0 Hz, 1H), 4.96 (dt, J=10.4 Hz, J=1.2 Hz, 1H), 4.12-4.10 (m, 1H), 4.04-4.02 (m, 1H), 3.52-3.49 (m, 1H), 3.17-3.09 (m, 2H), 2.35-2.31 (m, 2H)

### Step-3: Synthesis of (S)-3-(2-hydroxyethoxy)-4-(trityloxy) butan-1-ol (4)

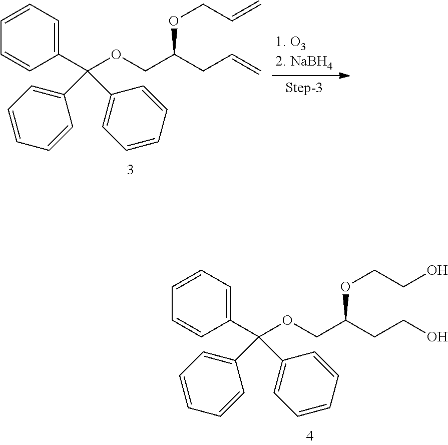

To a stirred solution of compound-3 (100 g, 0.26 mol) in MeOH:DCM (1:1) (800 mL) was bubbled ozone gas for 18 h at −45° C. After completion of the reaction (monitored by TLC), it was poured into a solution of sodium borohydride (21.5 g, 0.57 mol) in 0.5 N NaOH solution (370 mL) at 0° C. The resulting reaction mixture was allowed to stir at RT for 16 h. After completion of the reaction (monitored by TLC), quenched with 1 N HCl solution, until pH 6-7. Then resulting solution was extracted with ethyl acetate (750 mL). Separated the organic layer, dried over sodium sulphate, filtered and evaporated under vacuum to afford crude compound, which was further purified by silica gel column chromatography (100-200 mesh), eluting with 20-25% ethyl acetate in hexane. The pure fractions were collected and evaporated to afford the desired compound-4 as a yellow color gummy liquid (58 grams, 57% yield). ¹H NMR (400 MHz, DMSO-d₆): δ 7.42-7.40 (m, 6H), 7.34 (t, J=7.6 Hz, 6H), 7.28-7.24 (m, 3H), 4.60 (t, J=5.6 Hz 1H), 4.36 (t, J=5.6 Hz, 1H), 3.60-3.56 (m, 2H), 3.52-3.48 (m, 2H), 3.44-3.41 (m, 3H), 2.99-2.97 (m, 2H), 1.61-1.56 (m, 2H).

### Step-4: Synthesis of (S)-2-((4-((methylsulfonyl)oxy)-1-(trityloxy)butan-2-yl)oxy)ethyl methanesulfonate (5)

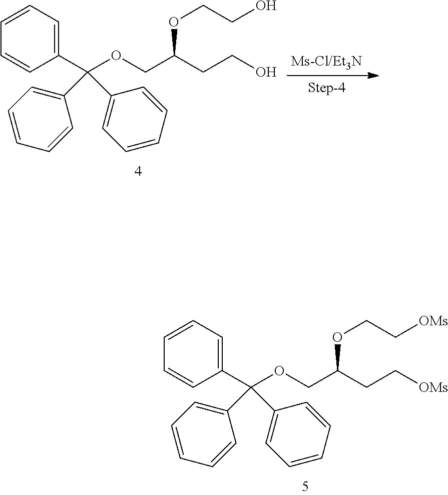

To a stirred solution of Compound-4 (60 g, 0.15 mol), in DCM (1000 mL) was added triethyl amine (66 mL, 0.47 mmol) at 0° C. and stirred for 15 min, followed by the addition of methane sulfonyl chloride (32.0 mL, 0.41 mmol). The resulting reaction mixture was stirred for 2 h at 0° C., (reaction monitored by TLC), quenched with sat. ammonium chloride solution (600 mL). Separated the organic layer, dried over sodium sulphate, filtered and evaporated under vacuum (<25° C.) to afford crude compound, which was suspended in a 1:1 mixture of ethyl acetate and heptane (600 mL) and evaporated under vacuum to give solid. The obtained solid compound was suspended in 1:1 mixture of ethyl acetate and heptane (600 mL), stirred for 30 min, filtered, washed the solid with heptane (80 mL) and dried under vacuum to afford compound-5 as a cream color solid (88 grams, 100% crude yield). ¹H NMR (400 MHz, DMSO-d₆): δ 7.42-7.39 (m, 5H), 7.37-7.31 (m, 5H), 7.29-7.23 (m, 3H), 7.22-7.18 (m, 2H), 4.34-4.22 (m, 4H), 3.84-3.83 (m, 1H), 3.69-3.64 (m, 2H), 3.17 (s, 31H), 3.13 (s, 3H), 3.09-3.06 (m, 1H), 3.04-3.02 (m, 1H), 1.88-1.85 (m, 2H).

### Step-5: Synthesis of (12E,32E,7S)-21-methyl-7-((trityloxy)methyl)-22,25-dihydro-11H,21H,31H-6-oxa-1,3(3,1)-diindola-2(3,4)-pyrrolacyclononaphane-22,25-dione (7)

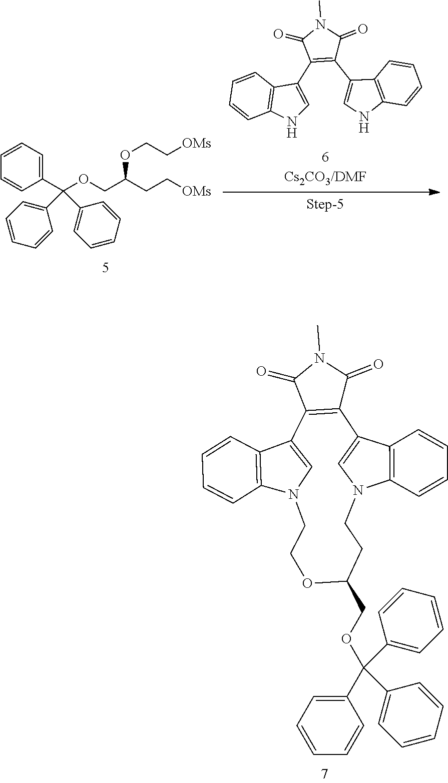 [SMILES: CN1C(=O)C(c2c[nH]c3ccccc23)=C(c2c[nH]c3ccccc23)C1=O ; CS(=O)(=O)OCCO[C@@H](CCOS(C)(=O)=O)COC(c1ccccc1)(c1ccccc1)c1ccccc1 ; CN1C(=O)C2=C(C1=O)c1cn(c3ccccc13)CC[C@@H](COC(c1ccccc1)(c1ccccc1)c1ccccc1)OCCn1cc2c2ccccc21]

To a stirred solution of Compound-6 (41.5 g, 0.12 mol) in DMF (850 mL) was added cesium carbonate (86.0 g, 0.26 mol) and the reaction mixture was heated to 100° C., then was added Compound-5 (85.0 g (crude), 0.15 mol) dropwise at same temperature. The resulting reaction mixture was stirred at 100° C. for 24 h. After completion of the reaction (monitored by TLC), cooled to 50° C., celite (25 g) was added and stirred for 15 min. The reaction mass was filtered on celite and the filtrate was partitioned between ethyl acetate (800 mL) and water (400 mL). Separated the organic layer, dried over sodium sulphate, filtered and evaporated under vacuum to afford crude compound, which was further purified by silica gel column chromatography (100-200 mesh), eluting with 25-30% ethyl acetate in hexane. The pure fractions were collected and evaporated to afford the desired compound-7 as a brick red solid (55 grams, 51% yield). ¹H NMR (400 MHz, DMSO-d₆): δ 7.83 (d, J=7.6 Hz 1H), 7.75 (d, J=8.0 Hz 1H), 7.49 (d, J=8.4 Hz 1H), 7.45 (s, 1H), 7.41 (s, 1H), 7.34-7.26 (m, 10H), 7.25-7.22 (m, 6H), 7.18-7.15 (m, 2H), 7.12-7.06 (m, 2H), 4.25-4.24 (m, 1H), 4.17-4.04 (m, 3H), 3.71-3.67 (m, 1H), 3.55-3.50 (m, 1H), 3.31-3.30 (m, 1H), 3.07 (s, 3H), 3.05-3.00 (m, 2H), 2.10-2.07 (m, 1H), 2.02 (m, 1H).

### Step-6: Synthesis of (12E,32E,7S)-7-((trityloxy)methyl)-22,25-dihydro-11H,31H-6-oxa-1,3(3,1)-diindola-2(3,4)-furanacyclononaphane-22,25-dione (8)

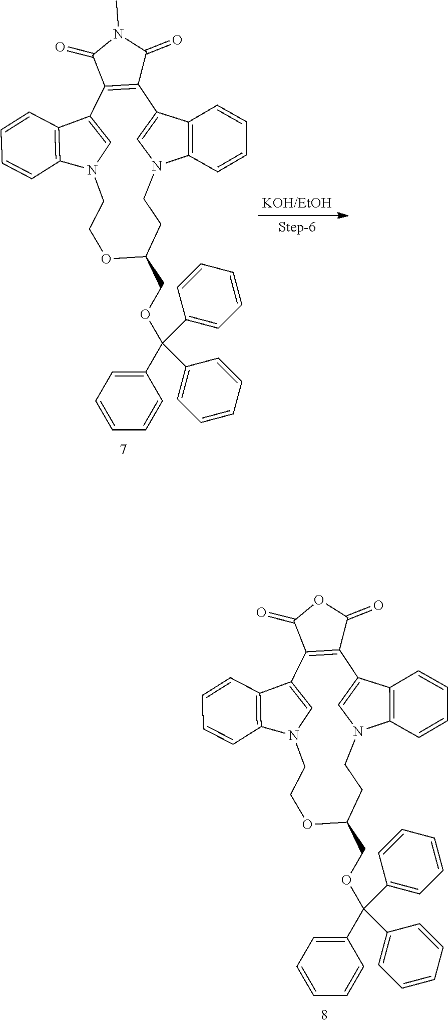 [SMILES: CN1C(=O)C2=C(C1=O)c1cn(c3ccccc13)CC[C@@H](COC(c1ccccc1)(c1ccccc1)c1ccccc1)OCCn1cc2c2ccccc21 ; O=C1OC(=O)C2=C1c1cn(c3ccccc13)CCO[C@H](COC(c1ccccc1)(c1ccccc1)c1ccccc1)CCn1cc2c2ccccc21]

To a stirred solution of compound-7 (85.0 g, 0.12 mol) in ethanol (850 mL) was added potassium hydroxide (68.0 g, 1.22 mol) and heated to 80° C. The resulting reaction mixture was stirred for 24 h. After completion of the reaction (monitored by TLC), the reaction mixture was evaporated under vacuum to give residue, which was partitioned between DCM (850 mL) and 20% citric acid solution (450 mL). Separated the organic layer, dried over sodium sulphate, filtered and evaporated under vacuum to afford crude compound-8 as a dark brown solid (62 grams, 74% yield). ¹H NMR (400 MHz, DMSO-d₆): δ 7.88 (d, J=7.6 Hz 1H), 7.82 (d, J=7.6 Hz 1H), 7.65 (d, 1=2.0 Hz 2H), 7.55 (d, 1=8.0 Hz 1H), 7.41 (d, 1=7.6 Hz 1H), 7.34-7.26 (m, 12H), 7.25-7.20 (m, 5H), 7.19-7.13 (m, 2H), 4.33-4.28 (m, 1H), 4.20-4.06 (m, 3H), 3.73-3.69 (m, 1H), 3.58-3.54 (m, 1H), 3.09-3.07 (m, 2H), 2.17-2.12 (m, 1H), 2.01-1.97 (m, 1H). (extra protons in the aromatic region, not included)

### Step-7: Synthesis of (12E,32E,7S)-7-((trityloxy)methyl)-22,25-dihydro-11H,21H,31H-6-oxa-1,3(3,1)-diindola-2(3,4)-pyrrolacyclononaphane-22,25-dione (9)

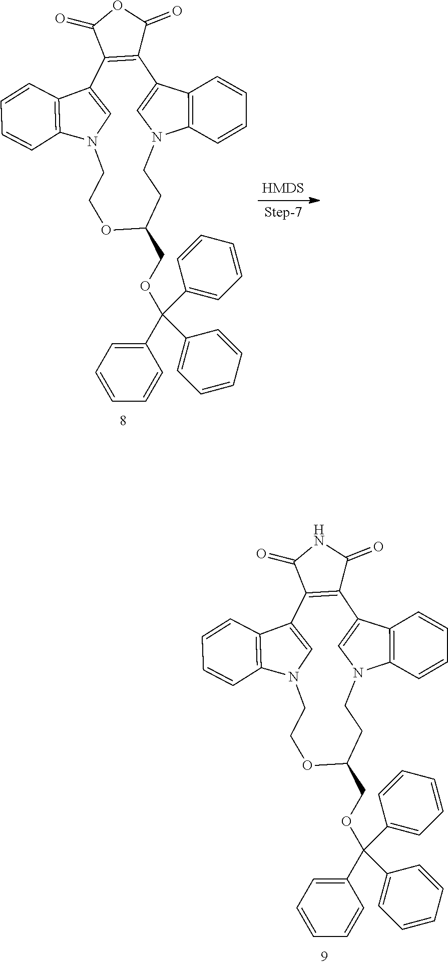 [SMILES: O=C1OC(=O)C2=C1c1cn(c3ccccc13)CCO[C@H](COC(c1ccccc1)(c1ccccc1)c1ccccc1)CCn1cc2c2ccccc21 ; O=C1NC(=O)C2=C1c1cn(c3ccccc13)CCO[C@H](COC(c1ccccc1)(c1ccccc1)c1ccccc1)CCn1cc2c2ccccc21]

To a stirred solution of Compound-8 (95.0 g, 0.25 mol) in DMF (950 mL) was added HMDS (294.0 mL, 2.47 mol), methanol (6.0 mL) and heated to 80° C. The reaction mixture was stirred for 5 h, at 80° C. After completion of the reaction (monitored by TLC), cooled to RT and quenched with 1N HCl solution (950 mL) and extracted with DCM (1500 mL). Separated the organic layer, dried over sodium sulphate, filtered and evaporated under vacuum to afford crude compound (84 g), which was further purified by silica gel column chromatography (100-200 mesh), eluting with 20-25% ethyl acetate in hexane. The pure fractions were collected and evaporated under vacuum to afford the desired compound-9 as a purple solid (70 grams, 74% yield). ¹H NMR (400 MHz, DMSO-d₆): δ 10.91 (s, 1H), 7.81 (d, J=8.0 Hz 1H), 7.73 (d, J=8.0 Hz 1H), 7.48 (d, J=8.4 Hz 2H), 7.43 (s, 1H), 7.39 (s, 1H), 7.33-7.23 (m, 12H), 7.23-7.21 (m, 3H), 7.18-7.14 (m, 2H), 7.11-7.06 (m, 2H), 4.27-4.23 (m, 1H), 4.13-4.00 (m, 3H), 3.70-3.67 (m, 1H), 3.55-3.47 (m, 1H), 3.33-3.26 (m, 1H), 3.02-2.99 (m, 2H), 2.13-2.08 (m, 1H), 2.01-1.98 (m, 1H).

### Step-8: Synthesis of (12E,32E,7S)-7-(hydroxymethyl)-22,25-dihydro-11H,21H,31H-6-oxa-1,3(3,1)-diindola-2(3,4)-pyrrolacyclononaphane-22,25-dione (10)

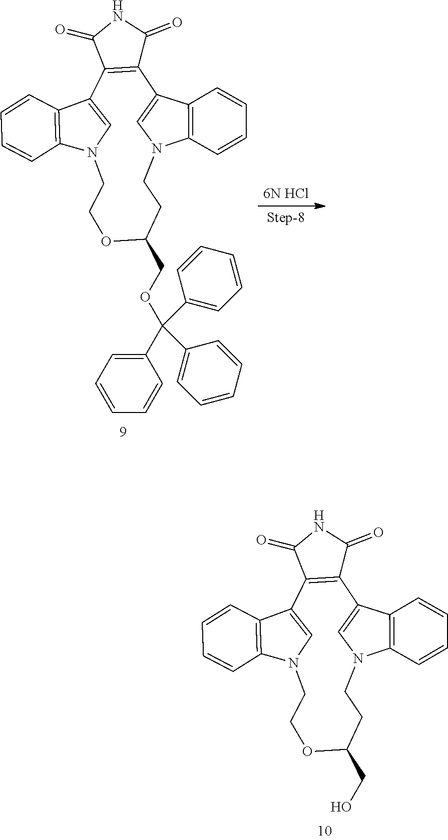

To a stirred solution of compound-9 (70.0 g, 0.18 mol) in ethanol (700 mL) was added 6N HCl (700 mL) at RT. The resulting reaction contents were heated to 80° C. for 3 h. After completion of the reaction (monitored by TLC), cooled to RT, stirred for 1 h, filtered the resulting solid and washed with water (350 mL), dried under vacuum at 45° C. to afford Compound-10 as a purple solid (40 grams, 88% crude yield). ¹H NMR (400 MHz, DMSO-d₆): δ 10.92 (s, 1H), 7.82 (d, J=7.6 Hz 1H), 7.78 (d, J=7.6 Hz 1H), 7.53 (d, J=8.0 Hz, 1H), 7.51 (s, 1H), 7.46 (d, J=8.4 Hz, 1H), 7.45 (s, 1H), 7.25-7.22 (m, 2H), 7.13-7.10 (m, 2H), 4.69 (t, J=5.2 Hz 1H), 4.35-4.33 (m, 1H), 4.24-4.15 (m, 3H), 3.91-3.87 (m, 1H), 3.65-3.60 (m, 1H), 3.53-3.49 (m, 1H), 3.43-3.39 (m, 1H), 2.09-2.07 (m, 1H), 1.98-1.97 (m, 1H).

### Step-9: Synthesis of ((12E,32E,7S)-22,25-dioxo-22,25-dihydro-11H,21H,31H-6-oxa-1,3(3,1)-diindola-2(3,4)-pyrrolacyclononaphane-7-yl)methyl methanesulfonate (11)

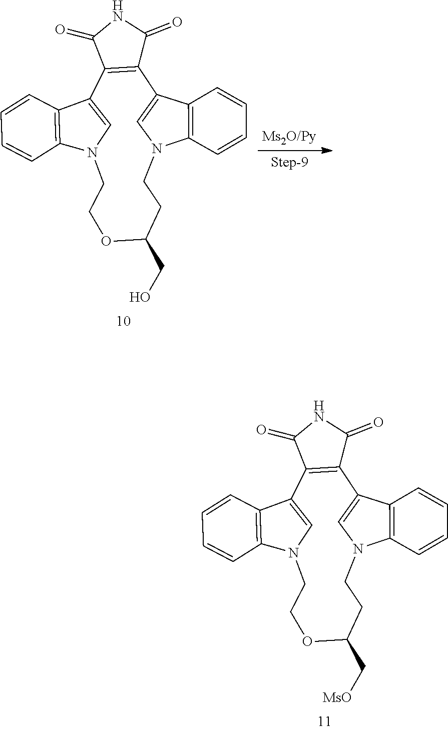

To a stirred solution of compound-10 (39.0 g, 0.09 mol) in THF (400 mL) was added pyridine (33.2 mL, 0.39 mol) at RT, stirred for 20 min, then methane sulfonic anhydride (46.0 g, 0.26 mol) was added into the reaction at RT. The resulting reaction mixture was stirred for 4 h. After completion of the reaction (monitored by TLC), partitioned the reaction between ethyl acetate (100 mL) and water (50 mL), separated the organic layer, dried over sodium sulphate, filtered and evaporated under vacuum to give crude compound (37.0 g), which was further purified by silica gel column chromatography (100-200 mesh), eluting with DCM. The pure fractions were collected and evaporated under reduced pressure to afford the desired compound-11 as a purple color solid (30 grams, 65% yield). ¹H NMR (400 MHz, DMSO-d₆): δ 10.92 (s, 1H), 7.83 (d, J=7.6 Hz 1H), 7.78 (d, J=7.6 Hz 1H), 7.54 (d, J=8.4 Hz, 1H), 7.52 (s, 1H), 7.48 (d, J=8.4 Hz, 1H), 7.46 (s, 1H), 7.22-7.17 (m, 2H), 7.14-7.10 (m, 2H), 4.44-4.38 (m, 2H), 4.22-4.14 (m, 4H), 3.93-3.90 (m, 1H), 3.66-3.61 (m, 1H), 3.17 (s, 3H), 2.19-2.14 (m, 1H), 2.03-1.98 (m, 1H).

### Step-10: Synthesis of (12E,32E,7S)-7-((methylamino)methyl)-22,25-dihydro-11H,21H,31H-6-oxa-1,3(3,1)-diindola-2(3,4)-pyrrolacyclononaphane-22,25-dione hydrochloride (Compound-1)

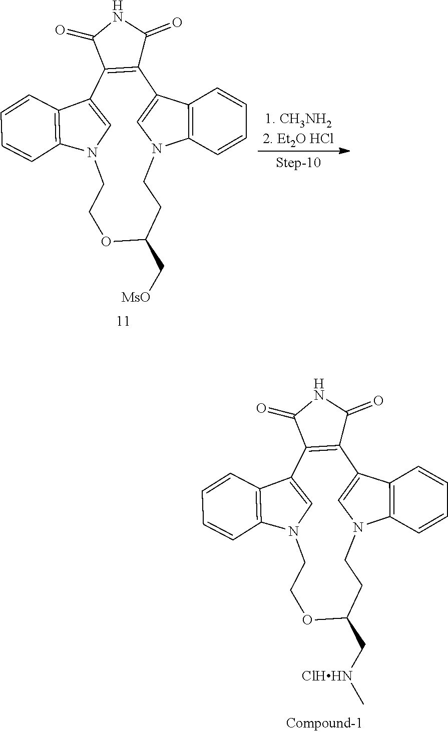

To a stirred solution of compound-11 (10.0 g, 0.019 mol) in THF (400 mL) was added 2M Methyl amine in THF (400 mL) at −40° C., in an auto-clave. Gradually heated the reaction to 70° C. and stirred for 24 h. After completion of the reaction (monitored by TLC), evaporated under vacuum to obtain crude compound (12.0 g). This batch was combined with 4 additional batches of the same scale giving 60.0 g of crude product. The 60 grams were purified by silica gel column chromatography (230-400 mesh, 2% MeOH/DCM). The pure fractions were collected and concentrated to give the desired compound-1 free-base (22.0 g) as a red solid. The free-base was suspended in diethyl ether (220 mL) and cooled to 0° C. Ethanolic HCl (33 mL) was added at 0° C. The resulting suspension was stirred at 0° C. for 30 min, filtered, washed with diethyl ether (50 mL) and dried under vacuum at 40° C. for 1 h to afford Compound-1 as a brick red color solid (16.9 grams, 36% yield). ¹H NMR (400 MHz, DMSO-d₆): δ 10.93 (s, 1H, exchanged in D₂O), 8.72-8.71 (m, 2H, exchanged in D₂O), 7.81 (t, J=8.0 Hz, 2H), 7.55 (d, J=8.0 Hz 1H), 7.49 (s, 2H), 7.47 (d, J=8.4 Hz, 1H), 7.23 (t, J=7.2 Hz, 2H), 7.14 (t, J=7.2 Hz, 2H), 4.46-4.41 (m, 1H), 4.33-4.25 (m, 2H), 4.15-4.10 (m, 1H), 3.86-3.84 (m, 1H), 3.73-3.71 (m, 1H), 3.62 (t, J=9.2 Hz, 1H), 3.27-3.24 (m, 1H), 3.01-2.98 (m, 1H), 2.53 (t, J=5.6 Hz, 3H), 2.22-2.20 (m, 1H), 2.06-2.03 (m, 1H).

### Example 2. Kinase Assay

Ruboxistaurin was purchased from a commercial laboratory and N-desmethyl ruboxistaurin was synthesized as in Example 1. Kinase reactions were carried out in a 384-well format. Reaction conditions included 0.25 ng GSK3α or GSK3β (0.62 and 0.68 nM final enzyme concentration, respectively), 0.25 μg GSK substrate, ATP (19 or 12 μM for GSK3α or β respectively), and a buffer of 50 mM Tris (with pH 7.5, 5 mM MgCl2, 0.01% Brij-35, and 3 mM DTT). Compound or 1% DMSO were added. A 5× stock of the above buffer was made up (without the DTT) and stored at room temperature.

Two solutions were made. The first contained 1× buffer and 2× enzyme and substrate. The second contained 1× buffer and 2×ATP. Each plate was loaded 2.5 μL of the first solution, then compound was added followed by a 15-minute incubation at room temperature, and then 2.5 μL of the second solution was added (followed by a pulse spin). Plates were then incubated at room temperature for 60 minutes (in the dark using another plate as a lid). To stop the reaction and deplete the remaining ATP, 5 μL of the ADP-Glo reagent was added to each well, before the plate was left at room temperature for a further 40 minutes. To detect ADP production, 10 μL of the ADP-Glo Kinase detection substrate was added to each well. After 5 to 30 minutes, the plate was read for luminescence.

### Example 3. Microsomal Stability

A master solution was prepared with 100 mM phosphate buffer, 5 mM MgCl₂ solution, and 0.5 mg/mL human microsomes. 40 μL of 10 mM NADPH solution was added to each well to produce a final concentration of 1 mM NADPH. The mixture was pre-warmed at 37° C. for 5 minutes. The negative control samples were prepared by replacing NADPH solutions with 40 μL of ultra-pure H₂O. Samples with NADPH were prepared in duplicate. Negative controls were prepared in singlet. The reaction was started with the addition of 2 μL of 200 μM control compound or test compound solutions. Verapamil was used as positive control. The final concentration of test compound or control compound was 1 μM.

Aliquots of 50 μL were taken from the reaction solution at 0, 15, 30, 45 and 60 minutes. The reaction was stopped by the addition of 4 volumes of cold acetonitrile with IS (100 nM alprazolam, 200 nM imipramine, 200 nM labetalol and 2 μM ketoprofen). Samples were centrifuged at 3, 220 g for 40 minutes. An aliquot of 90 μL of the supernatant was mixed with 90 μL of ultra-pure H₂O and then used for LC-MS/MS analysis. Peak areas were determined from extracted ion chromatograms.

### Example 4. Blood/Brain Barrier Penetration

Male Sprague Dawley (SD) Rats were administered 1 mg/kg of N-desmethyl ruboxistaurin intravenously (via the tail vein). Blood samples were collected from 3 animals at 0.25, 1-, and 4-hours post-dose. Brain samples were collected from 9 other rats, 3 each at 0.25, 1-, and 4-hours post-dose. Brain samples were weighted and homogenized with phosphate buffered saline. Samples (20 μL) were added to 200 μL of acetonitrile containing mixture for precipitation and then vortexed for 30 s. After centrifugation at 4 degrees Celsius, 4000 rpm for 15 min, the supernatant was diluted with ultrapure H₂O at a ratio of 1:2, then 15 μL of supernatant was injected into the LC/MS/MS system (liquid chromatography with tandem mass spectrometry) to analyze levels of N-desmethyl ruboxistaurin.

### Example 5. Ultrasonic Vocalizations in Rats

98 male Wistar rats were acclimated to the testing facility and handled daily (10 min/day) for 7 days prior to initiating the experiment. Male Wistar rats (˜200 g) were used in this study: 8 rats each in 5 groups (lithium and 0, 10, 30, 100 mg/kg N-desmethyl ruboxistaurin prior to administration of dextroamphetamine, or D-AMP). There were also 9 rats that did not receive D-AMP. Rats were placed in white Plexiglas boxes (50×50×50 cm) and videotaped for 10 minutes to acclimate them to the test apparatus. Ultrasonic vocalizations (USV) were recorded with a microphone mounted 45 cm above the open field box to establish baseline, non-treatment related USV responses (50-kHz calls) in each rat. The next day, rats were injected with saline (1 mL/kg body weight, IP) and immediately placed into open field-testing boxes. USVs were recorded for 10 min. This data served as the basis of selecting equal groups for the drug test. On day 3, rats were administered the positive treatment control (100 mg/kg lithium carbonate in saline administered subcutaneously, or 0, 10, 30, or 100 mg/kg N-desmethyl ruboxistaurin intraperitoneally (IP). D-amphetamine (in saline), given IP at 2.5 mg/kg, was administered 60 minutes later and rats were immediately placed into the open field-testing boxes. USVs were recorded for 10 min.

What is claimed is:

1. N-desmethyl ruboxistaurin or a pharmaceutically acceptable salt thereof, for use in treating a disorder for which GSK3β inhibition is clinically useful, wherein the use comprises administering to a subject in need thereof a therapeutically effective dose of the N-desmethyl ruboxistaurin or a pharmaceutically acceptable salt thereof,
wherein the subject 1) has never taken ruboxistaurin, or 2) has taken ruboxistaurin and has experienced adverse effects, or 3) shows a prolonged QT interval, or 4) has been shown to have elevated plasma levels of ruboxistaurin, or 5) has the potential to receive drugs that might interfere with the metabolism of ruboxistaurin, or 6) might require higher doses of ruboxistaurin and there is a concern for adverse effects, QT prolongation, or adverse drug interactions,
and wherein the subject has a neurological disease and/or psychiatric disorder.

2. The N-desmethyl ruboxistaurin or pharmaceutically acceptable salt thereof for use according to claim 1, wherein the disease/disorder is selected from Alzheimer's disease, frontotemporal dementia, behavioral complications of dementia, bipolar disorder, depression, schizophrenia, Parkinson's disease, neuroinflammation, autism spectrum disorder, Fragile X syndrome, Pitt Hopkins syndrome, Rett syndrome, traumatic brain injury, stroke, acute spinal cord injury, amyotrophic lateral sclerosis (ALS), multiple sclerosis (MS), neurofibromatosis type 1, neuronal ceroid lipofuscinosis, chronic pain, neuropathic pain, chemotherapy-induced neuropathy, and chemotherapy-induced cognitive impairment.

3. The N-desmethyl ruboxistaurin or pharmaceutically acceptable salt thereof for use according to claim 1, wherein N-desmethyl ruboxistaurin, or a pharmaceutically acceptable salt thereof is administered in an amount of 32 to 320 mg once daily, or 16 to 160 mg twice daily.

4. The N-desmethyl ruboxistaurin or pharmaceutically acceptable salt thereof for use according to claim 1, wherein N-desmethyl ruboxistaurin, or a pharmaceutically acceptable salt thereof is administered in combination with lithium.

5. The N-desmethyl ruboxistaurin or pharmaceutically acceptable salt thereof for use according to claim 4, wherein the subject is non-responsive to lithium.

6. The N-desmethyl ruboxistaurin or pharmaceutically acceptable salt thereof for use according to claim 4, wherein the subject is lithium responsive.

7. The N-desmethyl ruboxistaurin or pharmaceutically acceptable salt thereof for use according to claim 4, wherein lithium is administered at a sub-effective dose based on monotherapy, and wherein N-desmethyl ruboxistaurin or a pharmaceutically acceptable salt thereof is administered at a sub-effective dose based on monotherapy.

8. The N-desmethyl ruboxistaurin or pharmaceutically acceptable salt thereof for use according to claim 7, wherein the sub-effective dose of lithium is about 60 mg to about 600 mg once daily, or about 30 mg to about 300 mg twice daily.

9. The N-desmethyl ruboxistaurin or pharmaceutically acceptable salt thereof for use according to claim 5, wherein a sub-effective dose of N-desmethyl ruboxistaurin, or pharmaceutically acceptable salt thereof is administered in about 8 to about 32 mg once daily, or about 4 to about 16 mg twice daily.

10. N-desmethyl ruboxistaurin or a pharmaceutically acceptable salt thereof for use according to claim 1, wherein N-desmethyl ruboxistaurin, or a pharmaceutically acceptable salt thereof is administered in combination with valproic acid, lamotrigine, quetiapine, olanzapine, risperidone, aripiprazole, lurasidone, lumateperone, cariprazine, asenapine, carbamazepine, xanomeline-trospium, iloperidone, or combinations thereof.

11. N-desmethyl ruboxistaurin or a pharmaceutically acceptable salt thereof, for use in establishing a diagnosis of bipolar disorder or other condition where GSK3β inhibition is clinically useful, wherein the use comprises administering to a subject to be evaluated a therapeutically effective dose of N-desmethyl ruboxistaurin or a pharmaceutically acceptable salt thereof, and evaluating the subject's clinical response.

12. N-desmethyl ruboxistaurin or a pharmaceutically acceptable salt thereof, for use in treating a subject with Alzheimer's disease, frontotemporal dementia, behavioral complications or dementia, bipolar disorder, or depression, and the condition is characterized by evidence of elevated GSK3β, wherein the use comprises administering to the subject a therapeutically effective dose of N-desmethyl ruboxistaurin or a pharmaceutically acceptable salt thereof, and evaluating and monitoring the subject using positron emission tomography (PET) or serology.

13. N-desmethyl ruboxistaurin or pharmaceutically acceptable salt thereof, for use in establishing a diagnosis of bipolar disorder or other condition where GSK3β inhibition is clinically useful, wherein the use comprises administering to a subject to be evaluated a therapeutically effective dose of N-desmethyl ruboxistaurin or a pharmaceutically acceptable salt thereof, with a therapeutically effective dose of lithium, and evaluating the subject's clinical response.

14. The N-desmethyl ruboxistaurin or pharmaceutically acceptable salt thereof for use according to claim 13, wherein the dose of both N-desmethyl ruboxistaurin and lithium are sub-effective based on monotherapy.

15. N-desmethyl ruboxistaurin or pharmaceutically acceptable salt thereof, for use in treating a subject with Alzheimer's disease, frontotemporal dementia, behavioral complications of dementia, bipolar disorder, or depression, and the condition is characterized by elevated GSK3β beta activity, wherein the use comprises administering to the subject a therapeutically effective dose of N-desmethyl ruboxistaurin or a pharmaceutically acceptable salt thereof, and a therapeutically effective dose of lithium, and monitoring the subject using positron emission topography (PET).

16. The N-desmethyl ruboxistaurin or pharmaceutically acceptable salt thereof, for use according to claim 15, wherein the dose of both N-desmethyl ruboxistaurin and lithium are sub-effective based on monotherapy.
